(19) United States
(12) Patent Application Publication
(10) Pub. No.: US 2008/0194870 A1
(43) Pub. Date: Aug. 14, 2008

(54) Process for the Preparation of a Dicarboxylic Acid

(76) Inventors: Eit Drent, Amsterdam (NL); Rene Ernst, Amsterdam (NL); Willem Wade Jager, Amsterdam (NL); Cornelia Alida Krom, Amsterdam (NL)

Correspondence Address: SHELL OIL COMPANY, P O BOX 2463, HOUSTON, TX, 772522463, US

(21) Appl. No.: 11/884,079

(22) PCT Filed: Feb. 10, 2006
(86) PCT No.: PCT/EP06/50824

§ 371 Date: Apr. 10, 2008

(30) Foreign Application Priority Data
Feb. 11, 2005 (EP) 05101012.2

(51) Int. Cl. C07C 51/14

(52) U.S. Cl. 562/542

(57) ABSTRACT

A process for the preparation of a dicarboxylic acid, comprising the steps of (a) contacting a conjugated diene with carbon monoxide and water in the presence of a catalyst system including a source of palladium, a source of an anion and a bidentate phosphine ligand, to obtain a mixture comprising an ethylenically unsaturated acid product; (b) reacting the mixture obtained in step (a) further with carbon monoxide and water to obtain the dicarboxylic acid in admixture with the ethylenically unsaturated acid; (c) separating the dicarboxylic acid from a liquid filtrate comprising the catalyst system; and (d) recycling at least part of the obtained liquid filtrate to step (a).

### FIELD OF THE INVENTION

The present invention provides a process for the preparation of a dicarboxylic acid by carbonylation of a conjugated diene to obtain an ethylenically unsaturated acid, and subsequent carbonylation of the ethylenically unsaturated acid to obtain a dicarboxylic acid.

### BACKGROUND OF THE INVENTION

Carbonylation reactions of conjugated dienes are well known in the art. In this specification, the term carbonylation refers to the reaction of a conjugated diene under catalysis by a transition metal complex in the presence of carbon monoxide and water, as for instance described in EP-A-0284170, EP-A-1625109, U.S. Pat. No. 6,008,408 and WO 04/103948. An important feature for the effectiveness of all industrial scale processes that employ transition metal catalysts resides in the loss of catalyst with product or purge streams, which requires complex recovery steps, and the inactivation of catalyst in the reaction and recovery steps, which increases costs.

In U.S. Pat. No. 6,008,408, a process is disclosed for the hydrocarboxylation of 2- and 3-pentenoic acid to adipic acid by carbon monoxide and water, in the presence of a catalyst based on iridium and/or rhodium and at least one iodinated promoter. The obtained mixture of catalyst, pentenoic acid, reaction by-products and adipic acid is subjected to a refining operation consisting in removing volatile components from this mixture by distillation under reduced pressure, followed by crystallizing the adipic acid from a remaining concentrate in multiple crystallization steps. This complex process permits recovery of up to80% of the catalyst, which may be recycled to the carbonylation reaction. Alternatively, it is mentioned that the recovered crude rhodium or iridium catalyst could be employed in a carbonylation of 1,3-butadiene to 3-pentenoic acid, as set out in EP-A-0405433.

The disclosed process has the drawback of only achieving a limited selectivity for the desired adipic acid, while delivering a large number of by-products. As a result, the adipic acid is only obtained in limited purity, and hence requires a complex purification allowing only a limited amount of catalyst to be recovered. The repeated crystallization steps are time and energy consuming, and require cumbersome handling of solid crystals soaked with liquid. Moreover, all product and purge streams contain impurities due to the presence of an iodine promoter. Therefore, the described process is considered unsuitable for an industrial scale production, in particular under continuous operation.

Accordingly, there remained the need to provide for a process for the preparation of saturated dicarboxylic acids from a conjugated diene that allows simple recovery and recycling of the catalyst, thereby making the process suitable for industrial application.

It has now been found that the above identified process for the preparation of a saturated diacids product from a conjugated diene can be very effectively performed as set out below, which makes it particularly suited as a semi-continuous or continuous industrial scale process.

### SUMMARY OF THE INVENTION

Accordingly, the subject invention provides a process for the preparation of a dicarboxylic acid, comprising the steps of
(a) contacting a conjugated diene with carbon monoxide and water in the presence of a catalyst system including a source of palladium, a source of an anion and a bidentate phosphine ligand, to obtain a mixture comprising an ethylenically unsaturated acid product;
(b) reacting the mixture obtained in step (a) further with carbon monoxide and water to obtain the dicarboxylic acid in admixture with the ethylenically unsaturated acid;
(c) separating the dicarboxylic acid from a liquid filtrate comprising the catalyst system; and
(d) recycling at least part of the obtained liquid filtrate to step (a).

### FIGURES

FIG. 1 is a schematic representation of a preferred embodiment of the process according to the invention.

### DETAILED DESCRIPTION OF THE INVENTION

Applicants found that the high selectivity achieved in the carbonylation steps (a) and (b) in the presence of a palladium carbonylation catalyst yields the desired dicarboxylic acid in high purity, while permitting a simple recovery and recycling of the catalyst.

When for instance 1,3-butadiene is employed as conjugated diene, the adipic acid can precipitate or crystallize spontaneously from the reaction mixture obtained in step (b) in crystals of very high purity. This makes it possible to separate the dicarboxylic acid in a much more efficient manner than for instance in the process described in U.S. Pat. No. 6,008,408. The subject process has the further advantage that the catalyst system proves highly stable under the process conditions employed, and can therefore be directly recycled to step (a) after separation of the dicarboxylic acid, without the need for complex catalyst recovery steps, and without significant inactivation of catalyst in the reaction and recovery steps due to exposure to high temperature, as for instance described in EP-A-1036056 and EP-A-0284170. Moreover, the dicarboxylic acid product can be separated as such from the reaction mixture, and no esterification is required in order to prepare the more volatile mono-or diesters that can be removed by distillation from the catalyst stream, as described in EP-A-0284170.

In step (a) of the subject process, it was found that conjugated dienes have the tendency to reversibly form allylic alkenyl esters with any carboxylic acid present in the reaction mixture, in particular under catalysis by the carbonylation catalyst. Depending on the reaction conditions, these alkenyl esters can be formed in substantial amounts.

Without wishing to be bound to any particular theory, it is believed that the formation of the esters from the conjugated diene and the ethylenically unsaturated acid product is an equilibrium reaction catalyzed by the carbonylation catalyst, albeit at a comparatively slow rate. The presence of a high diene concentration, as well as an increasing amount of ethylenically unsaturated acid favours the formation of esters. In absence of catalyst, the equilibrium reaction becomes very slow, hence effectively freezing the equilibrium.

Since the alkenyl esters can be reverted into the conjugated diene and the ethylenically unsaturated acid, they are referred to as “reversible diene adducts” throughout the present specification. These “reversible diene adducts” were found to be remarkably stable in absence of the carbonylation catalyst.

In the case of 1,3-butadiene as conjugated diene, the “reversible diene adducts” are butenyl esters of carboxylic acids present in the reaction mixture, in particular butenyl esters of 2-, 3- and 4-pentenoic acid, and mixtures thereof. In the case of 1,3-butadiene as conjugated diene, the term ethylenically unsaturated acid describes 2-pentenoic acid, 3-pentenoic acid and 4-pentenoic acid, and mixtures thereof.

In step (a), the ratio (v/v) of diene and water in the feed can vary between wide limits and suitably lies in the range of 1:0.0001 to 1:500. However, it was found that addition of water in step (a) to the reaction medium in order to provide a higher concentration of this reactant, and hence to increase the reaction rate had the opposite effect, i.e. an increase of the water concentration resulted in a strongly decreased reaction rate. Hence, it appears that the polarity of the reaction mixture influences the reaction speed, i.e. the reaction of step (a) is favoured by a more apolar medium.

Therefore, preferably, in step (a), less than 5% by weight of water is present in the reactor, yet more preferably, less than 3% by weight of water, yet more preferably, less than 1% by weight of water, again more preferably less than 0.15% by weight of water, and most preferably less than 0.001% by weight of water (w/w) is present in the reactor, calculated on the total weight of reactants. Again more preferably, these water concentrations are continuously present only, in particular if the reaction is performed as semi-batch or as continuous process. The water concentration may be determined by any suitable method, for instance by a Karl-Fischer-titration.

The polarity of the reaction mixture may further be influenced by the selection of reaction medium. This may be achieved for instance by addition of an apolar solvent e.g. toluene. It was also found that if the diene feed contained alkenes and alkynes, since the amount of these apolar compounds was higher in the reaction medium at a constant level of conjugated diene, the overall medium was les polar, and the reaction equally proceeded faster.

The reaction rate towards the end of the reaction can be increased by an increase in reactor temperature; this however was found to reduce the catalyst lifetime.

During the carbonylation reaction in step (a), the reaction medium will be increasingly depleted of the conjugated diene towards the end of the reaction. It was observed in a batch reaction that the concentration of the conjugated diene only very slowly approached a minimum concentration, while not falling below this minimum concentration.

Without wishing to be bound to any particular theory, it is believed that this is due to the presence of reversible diene adducts, which only slowly revert back to the conjugated diene and the acid to which they stand in equilibrium, even under catalysis by the palladium carbonylation catalyst. Accordingly, the overall reaction rate becomes increasingly dependent on speed of the reversion of the reversible esters to conjugated diene.

In order to avoid arriving at a low concentration of conjugated diene, step (a) of the present process is preferably not allowed to proceed to full conversion of the conjugated diene and its reversible adducts, but only to partial conversion. Then any remaining conjugated diene and reversible adducts are preferably removed from the reaction mixture prior to, or during step (b).

In the case of the carbonylation of 1,3-butadiene, step (a) is preferably allowed to proceed to 95% of conversion based on moles of 1,3-butadiene converted versus moles of 1,3-butadiene fed. Yet more preferably, step (a) is allowed to proceed to 85% of conversion, again more preferably to 75% of conversion, again more preferably step to 65% of conversion, and yet again more preferably step (a) is allowed to proceed to 60% of conversion. Again more preferably, the reaction is conducted in such way, that the conversion of 1,3-butadiene in step (a) is in the range of from 30 to 60%, based on moles of 1,3-butadiene converted versus moles of 1,3-butadiene fed.

According to a preferred embodiment of the present process, the conjugated diene and reversible diene adducts are removed from the reaction medium obtained in step (a) prior to step (b) to avoid the slowing down of the reaction rate when a high conversion is approached. Thereby, carbon monoxide, conjugated diene and the reversible ester products are removed from the reactor, while at least part of the ethylenically unsaturated acid product and the catalyst system remain in the reactor. This may preferably be done by removal of the reversible diene adducts from the reaction mixture by an in-situ conversion, and simultaneous removal of the conjugated diene. The in-situ conversion may preferably be performed in the following manner: provided the conjugated diene is gaseous or has a low boiling point at ambient pressure, as for instance the case of 1,3-butadiene, the reaction mixture obtained in step (a) is brought near to atmospheric pressure, and then the conjugated butadiene is stripped from the reaction mixture under a gas flow, preferably a gas flow comprising carbon monoxide. The latter provides additional stability to the catalyst. In this way, the reversible diene adducts are forced to revert back into the conjugated diene and the ethylenically unsaturated acid, since constant removal of the conjugated diene with the gas stripping stream will move the equilibrium towards reversion. The gaseous stripping stream obtained comprising carbon monoxide and conjugated diene may then advantageously be returned to step (a).

Alternatively, the reversible diene adducts may be removed from the reaction mixture in a distillative operation. The removed obtained ester mixture, usually also comprising some ethylenically unsaturated acid and reaction by-products, is then either directly recycled to step (a), or may be converted in a separate conversion step in the presence of a suitable catalyst back into the conjugated diene and the ethylenically unsaturated acid. At this point in the process, any undesired side-products may advantageously be removed as well. For this conversion, the reversible diene adducts are contacted with a suitable catalyst before recycling the obtained conjugated diene and the unsaturated acid back to the process. Any catalyst suitable for the conversion may be applied, such as heterogeneous or homogeneous palladium catalysts. An example of a suitable palladium catalyst is the catalyst system as described for steps (a) and (b).

The reversible diene adducts usually have a boiling range below that of the unsaturated acid product.

If 1,3-butadiene is the conjugated diene, the distillative removal is preferably performed at a bottom temperature in range of from 70 to 150° C. and a pressure of from 1 to 30 kPa (10 to 300 mbar), yet more preferably at a bottom temperature in range of from 90 to 130° C. and a pressure of from 2.5 to 15 kPa, and most preferably, at a bottom temperature in the range of from 100 to 110° C. and at a pressure in the range of from 3 to 8 kPa. Although these pressures and temperatures are not critical, pressures of above 20 kPa should be avoided due to the high temperatures required, which may result in catalyst degradation, while pressures below 1 kPa will require specific equipment. The removal by distillation is more complex than the in-situ conversion, but the carbonylation catalyst of step (a) will be used more effectively.

The subject process permits to react a conjugated diene with carbon monoxide and water. The conjugated diene reactant has at least 4 carbon atoms. Preferably the diene has from 4 to 20 and more preferably from 4 to 14 carbon atoms. However, in a different preferred embodiment, the process may also be applied to molecules that contain conjugated double bonds within their molecular structure, for instance within the chain of a polymer such as a synthetic rubber. The conjugated diene can be substituted or non-substituted. Preferably the conjugated diene is a non-substituted diene. Examples of useful conjugated dienes are 1,3-butadiene, conjugated pentadienes, conjugated hexadienes, cyclopentadiene and cyclohexadiene, all of which may be substituted. Of particular commercial interest are 1,3-butadiene and 2-methyl-1,3-butadiene (isoprene).

In step (b), the mixture obtained in step (a) is pressurized again with carbon monoxide, and additional water is added as reactant for the carbonylation of the unsaturated acid product formed in step (a) is converted to a dicarboxylic acid under addition of carbon monoxide and water.

It was found that the reaction of the formed ethylenically unsaturated carboxylic acid to a diacid proceeds at an increased rate if the polarity of the medium is increased with respect to step (a). Therefore preferably, the water concentration throughout step (b) is higher as compared to step (a). Accordingly, the present invention relates to a process wherein in step (b) the water concentration in the reaction medium is maintained within the range of from to 3 to 50%, preferably from 4 to 30%, more preferably from 5 to 25%, and most preferably from 5 to 10% (w/w), based on the amount of the total liquid reaction medium. Preferably, step (b) is performed as semi-batch or as continuous process, and more preferably, all of steps (a), (b), (c) and (d) are performed continuously.

In the case of the carbonylation of 1,3-butadiene, step (b) results in adipic acid product and in high purity. Adipic acid is a highly crystalline solid at ambient conditions. In the case that the process is conducted in pentenoic acid as solvent, adipic acid may begin to crystallize from the reaction mixture from a certain concentration and temperature onwards. If spontaneous crystallization in the reactor for step (b) is not desired, preferably step (b) is also only allowed to proceed until the liquid reaction medium comprises a saturated solution of adipic acid and/or any by-products at the reaction temperature in the liquid reaction medium.

Suitable sources of palladium for steps (a) and (b) include palladium metal and complexes and compounds thereof such as palladium salts; and palladium complexes, e.g. with carbon monoxide or acetyl acetonate, or palladium combined with a solid material such as an ion exchange resin. Preferably, a salt of palladium and a carboxylic acid is used, suitably a carboxylic acid with up to 12 carbon atoms, such as salts of acetic acid, propionic acid and butanoic acid, or salts of substituted carboxylic acids. A very suitable source is palladium (II) acetate.

Any bidentate diphosphine resulting in the formation of an active carbonylation catalyst with palladium may be used in the subject process. Preferably, a bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom due to the higher activity found with such catalysts in both reaction steps. Yet more preferably, R represents an aromatic bidentate bridging group that is substituted by one or more alkylene groups, and wherein the phosphino groups R¹R²P— and —PR³R⁴ are bound to the aromatic group or to the alkylene group due to the observed high stability of these ligands. Most preferably R¹, R², R³ and R⁴ are chosen in such way, that the phosphino group PR¹R² differs from the phosphino group PR³R⁴. A very suitable ligand is 1,2-bis(di-tert-butylphosphinomethyl)benzene. The ratio of moles of a bidentate diphosphine per mole atom of palladium preferably ranges from 0.5 to 50, more preferably from 0.8 to 10, yet more preferably from 0.9 to 5, yet more preferably in the range of 0.95 to 3, again more preferably in the range of 1 to 3, and yet most preferably it is in the range of from 1 to 2. In the presence of oxygen, slightly higher than stoichiometric amounts of ligand to palladium are beneficial.

The source of anions preferably is an acid, more preferably a carboxylic acid, which preferably serves both as catalyst component as well as solvent for the reaction. Again more preferably, the source of anions is an acid having a pKa above 2.0 (measured in aqueous solution at 18° C.), and yet more preferably an acid having a pKa above 3.0, and yet more preferably a pKa of above 3.6. Examples of preferred acids include carboxylic acids, such as acetic acid, propionic acid, butyric acid, pentanoic acid, pentenoic acid and nonanoic acid, the latter three being highly preferred as their low polarity and high pKa was found to increase the reactivity of the catalyst system. 2-, 3- and/or 4-pentenoic acid is particularly preferred in case the conjugated diene is 1,3-butadiene. Preferably the reaction is conducted in 2-, 3- and/or 4-pentenoic acid, since this was found to not only form a highly active catalyst system, but also to be a good solvent for all reaction components.

The molar ratio of the source of anions, and palladium is not critical. However, it suitably is between 2:1 and 10⁹:1 and more preferably between 10⁷:1 and 10:1, yet more preferably between 10⁶:1 and 10²:1, and most preferably between 10⁵:1 and 10²:1 due to the enhanced activity of the catalyst system. Very conveniently the acid corresponding to the desired product of the reaction can be used as the source of anions in the catalyst. The process may optionally be carried out in the presence of an additional solvent, however preferably the intermediate acid product serves both as source of anions and as reaction solvent. Usually amounts in the range of 10⁻⁸ to 10⁻¹, preferably in the range of 10⁻⁷ to 10⁻² mole atom of palladium per mole of conjugated diene are used, preferably in the range of 10⁻⁵ to 10⁻² mole atom per mole of conjugated diene. If the amount of catalyst is chosen at a level below 20 ppm, calculated on the total amount of liquid reaction medium, side reactions, in particular Diels-Alder reactions of the conjugated diene, become more prominent. In the case of 1,3-butadiene, the side-products formed include 4-vinyl cyclohexene (further referred to as VCH, being the adduct of two 1,3-butadiene molecules), and 2-ethyl cyclohexene carboxylic acid, further referred to as ECCA, which is the adduct of 1,3-butadiene and 2-pentenoic acid. The formation of ECCA is favoured if pentenoic acid also serves a solvent. When about 20 ppm of palladium catalyst were employed, ECCA was formed in up to 3% by weight on total products formed. An increase of the catalyst concentration to 200 ppm is expected to result in a reduction of to 0.3% by weight of ECCA, and an increase of the catalyst concentration to 1000 ppm is expected to resulting a reduction to 0.06% by weight. Accordingly, in steps (a) and (b), the carbonylation is preferably performed in the presence of at least 20 ppm of catalyst, more preferably in the presence of 100 ppm of catalyst, and most preferably in the presence of at least 500 ppm. Although this requires a larger amount of palladium to be employed, the catalyst may advantageously be recycled to the reaction of either step (a) or (b).

Examples of suitable catalyst systems for steps (a) and (b) as described above are those disclosed in EP-A-1282629, EP-A-1163202, WO2004/103948 and/or WO2004/103942.

The carbonylation reaction according to the present invention in steps (a) and (b) is carried out at moderate temperatures and pressures. Suitable reaction temperatures are in the range of 0-250° C., more preferably in the range of 50-200° C., yet more preferably in the range of from 80-150° C.

The reaction pressure is usually at least atmospheric pressure. Suitable pressures are in the range of 0.1 to 25 MPa (1 to 250 bar), preferably in the range of 0.5 to 15 MPa (5 to 150 bar), again more preferably in the range of 0.5 to 9.5 MPa (5 to 95 bar) since this allows use of standard equipment. Carbon monoxide partial pressures in the range of 1 to 9 MPa (10 to 90 bar) are preferred, the upper range of 5 to 9 MPa being more preferred. Again higher pressures require special equipment provisions, although the reaction would be faster since it was found to be first order with carbon monoxide pressure.

In the process according to the present invention, the carbon monoxide can be used in its pure form or diluted with an inert gas such as nitrogen, carbon dioxide or noble gases such as argon, or co-reactant gases such as ammonia.

Process steps (a) to (d) are preferably performed in a continuous operation. Steps (a) and (b) of the subject process are suitably performed in a single reactor suitable for gas-liquid reactions, or a cascade thereof, such as constant flow stirred tank reactor, or a bubble column type reactor, as for instance described in “Bubble Column Reactors” by Wolf-Dieter Deckwer, Wiley, 1992. A bubble column reactor is a mass transfer and reaction device in which in one or more gases are brought into contact and react with the liquid phase itself or with a components dissolved or suspended therein. Preferably, a reactor with forced circulation is employed, which are generally termed “ejector reactors”, or if the reaction medium is recycled to the reactor, “ejector loop reactors”. Such ejector reactors are for instance described in U.S. Pat. No. 5,159,092 and JP-A-11269110, which employ a liquid jet of the liquid reaction medium as a means of gas distribution and circulation.

The dicarboxylic acid may be isolated from the reaction mixture by various measures. Preferably, the dicarboxylic acid is isolated from the reaction mixture by crystallization of the diacid in the reaction mixture and separation of the dicarboxylic acid crystals from the remaining reaction mixture containing the catalyst. It has been found that the dicarboxylic acid crystals can be obtained in a high purity in only a few crystallization steps, making it an efficient method for the separation of the product from the catalyst and unreacted ethylenically unsaturated acid intermediate.

In the process according to the present invention, the carbon monoxide can be used in its pure form or diluted with an inert gas such as nitrogen, carbon dioxide or noble gases such as argon, or co-reactant gases such as ammonia. Alternatively, the carbon monoxide can be used diluted with hydrogen and/or carbon dioxide, as for instance in synthesis gas.

The mixture obtained in step (b) is subjected to separation in step (c). Any separation method suitable to separate the dicarboxylic acid from a liquid stream comprising the unsaturated acid and catalyst may be employed.

Preferably, the mixture is cooled, more preferably slowly cooled to ambient temperature to allow formation of seed crystals. Any known crystallization technique may be employed, although the purity of the adipic acid and the nature of the side products formed usually allow spontaneous crystallization. More preferably, (c) may be performed in a reactor specifically adapted for crystallization, for instance a stirred tank reactor with internal or external cooling.

Subsequently, the obtained crystals are separated from a liquid stream comprising the unsaturated acid and catalyst. This may be done by any suitable known separation method. Preferably the separation is done by filtration or centrifugation. The obtained liquid filtrate comprising the active catalyst system is then in step (d) at least in part recycled to step (a). Preferably, since more water is present in step (b), at least part of any water present in the liquid filtrate prior is removed prior to recycling to step (a) in order to achieve optimum concentrations. Optionally, undesired side products can advantageously be removed from the catalyst recycling stream at this point in the process. The obtained dicarboxylic acid may further be subjected to additional purification steps. This may be done by any useful purification method.

Alternatively, step (c) preferably is performed in a single crystallization reactor with continuous removal of the crystallized product. Yet more preferably, steps (b) and (c) are combined and performed done in a single reactor set-up that allows carbonylation, and continuous removal of the obtained crystal products.

The process according to the invention further preferably comprises the steps of (i) converting the dicarboxylic acid to its dichloride, and (ii) reacting the dicarboxylic acid dichloride with a diamine compound to obtain an alternating co-oligomer or co-polymer.

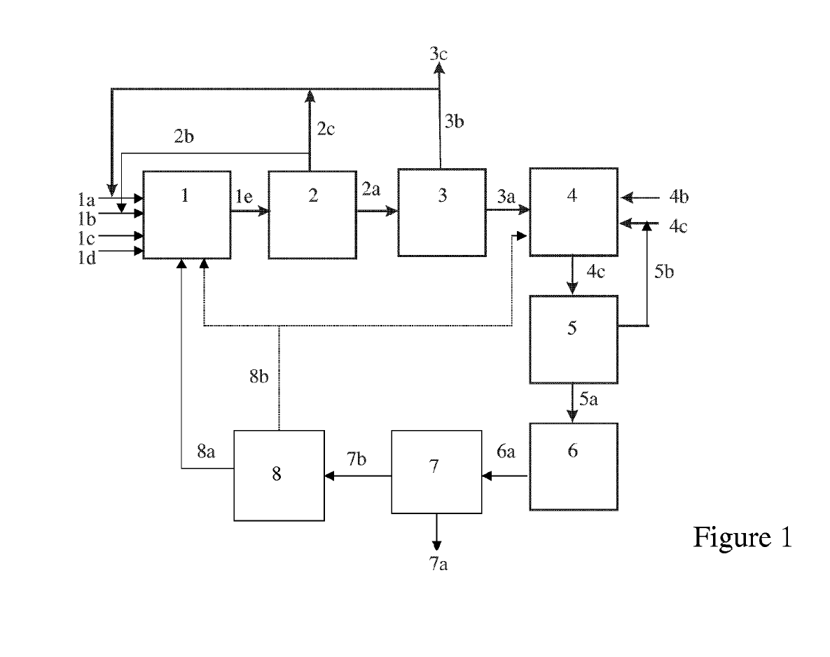

The invention will further be described by way of example with reference to FIG. 1, which is a schematic representation of a preferred embodiment of the process according to the present invention. FIG. 1 illustrates a process wherein a conjugated diene (1a), carbon monoxide (1b), water (1c) and a catalyst system including a source of palladium, a source of an anion and a bidentate phosphine ligand (1d) are supplied to a reactor (1). In this reactor (1), the conjugated diene is contacted with the carbon monoxide and water in the presence of a catalyst system including a source of palladium, a source of an anion and a bidentate phosphine ligand, to obtain a mixture comprising an ethylenically unsaturated acid product (1e). The mixture (1e) is then transported to vessel (2), where it is depressurized to obtain a depressurized mixture (2a). At this stage, optionally a stream of a normally gaseous conjugated diene (2c) and a stream of unreacted carbon monoxide (2b) may be separated from the mixture (1e). These may be recycled to reactor (1). The depressurized mixture (2a) is then transported into a stripping vessel (3), wherein a stream (3b) comprising the remaining conjugated diene and/or reversible diene adducts is removed to obtain a mixture (3a) comprising the ethylenically unsaturated acid product together with the catalyst system. The stream (3b) comprising the remaining conjugated diene and/or reversible diene adducts, optionally purged from Diels-Alder adducts formed from two molecules of conjugated diene (3c) can be recycled to the reactor (1), optionally in admixture with stream 2c.

The obtained depressurized and stripped mixture (3a) is transferred to a reactor (4), where it is reacted further under carbon monoxide pressure (4b) with additional water (4a) to obtain a stream (4c) comprising the saturated dicarboxylic acid in admixture with the ethylenically unsaturated acid and the catalyst system. The stream 4c is then depressurized (5), while remaining carbon monoxide (5b) is recycled to step (4), or may also be recycled to step (1).

The depressurized mixture (5a) is then cooled (6), and subjected to filtration (7) of the obtained crystals of the dicarboxylic acid, yielding crude adipic acid crystals (7a) and a liquid filtrate (7b). The liquid filtrate (7b) comprising the catalyst system in admixture with the ethylenically unsaturated acid is then optionally stripped (8) of surplus water, and the obtained dehydrated stream (8a) comprising the catalyst system in admixture with the ethylenically unsaturated acid is then recycled to step (1), or in total or in part to step (4). The separated of water (8b) may advantageously be returned to step (1) or step (4).

1. A process for the preparation of a dicarboxylic acid, comprising the steps of
(a) contacting a conjugated diene with carbon monoxide and water in the presence of a catalyst system including a source of palladium, a source of an anion and a bidentate phosphine ligand, to obtain a mixture comprising an ethylenically unsaturated acid product;
(b) reacting the mixture obtained in step (a) further with carbon monoxide and water to obtain the dicarboxylic acid in admixture with the ethylenically unsaturated acid;
(c) separating the dicarboxylic acid from a liquid filtrate comprising the catalyst system; and
(d) recycling at least part of the obtained liquid filtrate to step (a).

2. The process of claim 1, wherein in step (c), the mixture obtained in step (b) is cooled to precipitate the dicarboxylic acid, and subsequently the obtained precipitate is separated from the liquid filtrate comprising the catalyst system.

3. The process of claim 1, wherein at least part of any water present in the liquid filtrate is removed prior to recycling to step (a).

4. The process of claim 1, wherein in step (a) the water concentration is maintained at a range of from 0.001 to less than 3% by weight of water, calculated on the overall weight of the liquid reaction medium.

5. The process of claim 1, wherein in step (b) the water concentration is maintained at a range of from 3% to 50% by weight of water, calculated on the overall weight of the liquid reaction medium.

6. The process of claim 1, further comprising a step (e) of purifying the dicarboxylic acid filtrated from the reaction mixture in step (d).

7. The process of claim 1, wherein prior to step (b), reversible adducts of the conjugated diene and the ethylenically unsaturated acid formed in step (a) are removed from the reaction mixture by distillation, or by in-situ conversion into the conjugated diene and ethylenically unsaturated acid and wherein the conjugated diene is removed from the product.

8. The process of claim 1, wherein the ethylenically unsaturated acid product of step (a) is employed as solvent for the process.

9. The process of claim 1, wherein the bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom.

10. The process of claim 9, wherein R represents an aromatic bidentate bridging group that is substituted by one or more alkylene groups, and wherein the phosphino groups R¹R²P— and —PR³R⁴ are bound to the aromatic group or to the alkylene group.

11. The process of claim 9, wherein R¹, R², R³ and R⁴ are chosen in such way, that the phosphino group PR¹R² differs from the phosphino group PR³R⁴.

12. The process of claim 1, wherein the steps (a) to (d) are performed continuously.

13. The process of claim 1, wherein the conjugated diene is 1,3-butadiene.

14. The process of claim 1, further comprising the steps of
(i) converting the dicarboxylic acid to its dichloride, and
(ii) reacting the dicarboxylic acid dichloride with a diamine compound to obtain an alternating co-oligomer or co-polymer.
